(19) United States
(12) Patent Application Publication
(10) Pub. No.: US 2013/0226100 A1
(43) Pub. Date: Aug. 29, 2013

(54) INLINE LIQUID DRUG MEDICAL DEVICE WITH MANUALLY OPERATED ACTUATOR

(71) Applicant: Amir Lev, Kfar Saba (IL)

(72) Inventor: Amir Lev, Kfar Saba (IL)

(73) Assignee: MEDIMOP MEDICAL PROJECTS LTD., Ra'anana (IL)

(21) Appl. No.: 13/883,289

(22) PCT Filed: Oct. 27, 2011
(86) PCT No.: PCT/IL11/00834

§ 371 Date: May 3, 2013

(30) Foreign Application Priority Data
Nov. 4, 2010 (IL) 209102

(51) Int. Cl. A61M 5/32

(52) U.S. Cl. CPC A61M 5/32
USPC 604/246

(57) ABSTRACT

Inline liquid drug medical device having a longitudinal device axis, a housing with a flow control member rotatable within a transverse bore from a first flow control position for establishing flow communication between a first pair of ports for liquid drug reconstitution purposes to a second flow control position for establishing flow communication between a second pair of ports for liquid drug administration purposes, and a manually operated actuator for rotating the flow control member from its first flow control position to its second flow control position.

### FIELD OF THE INVENTION

The invention relates to inline liquid drug medical devices for liquid drug reconstitution and administration purposes.

### BACKGROUND OF THE INVENTION

Commonly owned U.S. Pat. No. 6,238,372 entitled Fluid Control Device illustrates and describes a fluid control device for use with a syringe and at least one medicinal vessel. The fluid control device includes a first port, a second port for receiving the syringe, a third port including an adaptor having a fluid conduit member extending into the interior of the medicinal vessel when attached thereto and a flow control member selectively disposable from a first flow control position enabling a flow path between a first pair of two ports and second flow control position enabling a flow path between a second pair of two ports. The flow control member is coupled to one of the ports for manipulation between its flow control positions.

Commonly owned PCT International Application No. PCT/IL2005/000376 entitled Liquid Drug Medical Devices and Needle Shield Removal Device and published under PCT International Publication No. WO 2005/105014 illustrates and describes a liquid drug medical device for liquid drug reconstitution and administration purposes, a vial adapter with elastomer tubing and a needle shield removal device. The liquid drug medical device has a longitudinal axis and is intended for use with a source of physiological solution and a medicinal vessel. The liquid drug medical device includes a body member having a first port for fluid connection with the source of physiological solution and a flow control member rotatably mounted in the body member about an axis of rotation co-directional with the longitudinal axis. The flow control member has a first major flow duct and a second major flow duct substantially parallel to and non-coaxial with the axis of rotation and respectively terminating at a second port, and a third port for administering the liquid drug. The liquid drug medical device further includes a manually rotatable adapter having a fluid conduit member with a proximal end in flow communication with the second port and a distal end extending into the medicinal vessel on its attachment to the adapter, and coupled to the flow control member for rotating same between a first flow control position for connecting the first port with the second port, and a second flow control position for connecting the first port with the third port.

Commonly owned PCT International Application No. PCT/US2008/070024 entitled Medicament Mixing and Injection Apparatus and published under PCT International Publication No. WO 2009/038860 illustrates and describes a mixing and injection apparatus including a needle and a needle base, a syringe attachment element and a mixing chamber engagement assembly including a needle chamber surrounding the needle and a first liquid conduit portion, sealed from the needle chamber and a mixing chamber engagement portion including a second liquid conduit portion communicating with the first liquid conduit portion and configured for communication with a mixing chamber. The syringe attachment element and the needle base are configured to permit liquid communication between an interior of the syringe and the first liquid conduit portion when the syringe attachment element and the needle base are in the first relative engagement orientation and to permit liquid communication between an interior of the syringe and the needle when the syringe attachment element and the needle base are in the second relative engagement orientation, axially separated from the first relative orientation along the injection axis.

Commonly owned PCT International Application No. PCT/IL2010/000915 entitled Inline Liquid Drug Medical Devices with Linear Displaceable Sliding Flow Control Member and published under PCT International Publication No. WO 2011/058548 illustrates and describes inline liquid drug medical devices having a longitudinal device axis, a housing with a linear displaceable sliding flow control member displaceable along a transverse bore from a first flow control position for establishing flow communication between a first pair of ports for liquid drug reconstitution purposes to a second flow control position for establishing flow communication between a second pair of ports for liquid drug administration purposes, and a manually operated actuating mechanism for applying a linear displacement force for urging the flow control member to slide along the bore from its first flow control position to its second flow control position.

### SUMMARY OF THE INVENTION

The present invention is directed toward inline liquid drug medical devices for use with a source of physiological fluid and a medicinal vessel for liquid drug reconstitution and administration purposes. The inline liquid drug medical devices include a housing having a longitudinal device axis and a vial adapter removably attached to the housing and detachable therefrom along a line of detachment co-directional with device axis. The housing has a bore transverse to the device axis for sealingly accommodating a flow control member rotatable from an initial first flow control position for liquid drug reconstitution purposes to a subsequent second flow control position for liquid drug administration purposes. The inline liquid drug medical devices include a manually operated actuator for engaging the flow control member for rotating same from its first flow control position to its second flow control position. The actuator, which resembles a key, is rotatably secured to the housing in an initial liquid drug reconstitution position, and is preferably removable from the housing following rotation to a subsequent liquid drug administration position. As part of a safety mechanism, the actuator has a safety hook which is latched to a safety catch on the vial adapter in the initial liquid drug reconstitution position. In this position, the housing and vial adapter are locked together and may not be separated. Rotating the actuator to the subsequent liquid drug administration position removes the safety hook from the safety catch, allowing for separation of the vial adapter from the housing for liquid drug administration purposes.

### BRIEF DESCRIPTION OF DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a pictorial representation of a syringe, a vial and an inline liquid drug medical device having a manually operated actuator for operating the device;

FIG. 2 is a perspective view of FIG. 1's device;

FIG. 3A is an exploded view of FIG. 1's device;

FIG. 3B is a top perspective view of a flow control member of FIG. 1's device;

FIG. 3C is a bottom perspective view of FIG. 3A's flow control member;

FIG. 3D is a front view of a housing of FIG. 1's device;

FIG. 4 is an enlarged longitudinal cross section of FIG. 1's device along line A-A in FIG. 1 showing its flow control member in a first flow control position for liquid drug reconstitution purposes;

FIG. 5 is an enlarged longitudinal cross section of FIG. 1's device along line A-A in FIG. 1 showing its flow control member in a second flow control position for liquid drug administration purposes; and

FIGS. 6A to 6G show the use of FIG. 1's device for liquid drug reconstitution and administration purposes.

### DETAILED DESCRIPTION OF PREFERRED EMBODIMENTS OF THE INVENTION

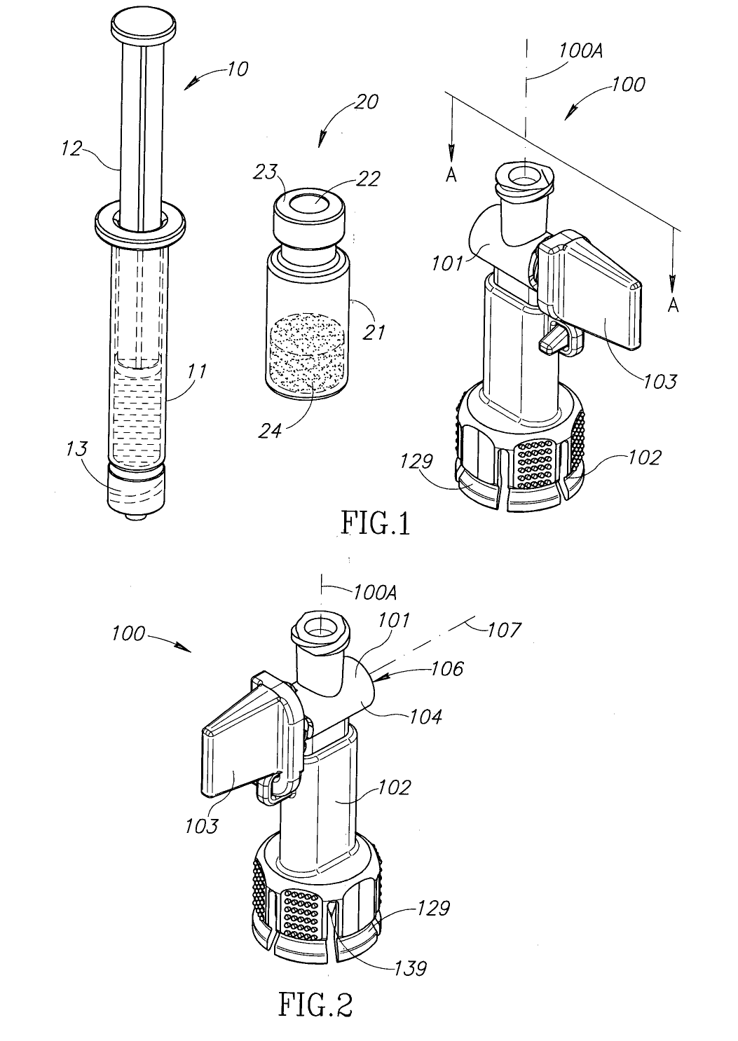

FIG. 1 shows a syringe 10 constituting a source of physiological fluid, a vial 20 constituting a medicinal vessel and an inline liquid drug reconstitution medical device 100 for use with the syringe 10 and the vial 20. The syringe 10 includes a barrel 11 with a plunger 12 and a male Luer lock connector 13. The syringe 10 can be formed with other types of male connectors. The vial 20 includes an open topped bottle 21 sealed by a vial stopper 22 capped by a band 23 or other suitable capping material. The vial 20 contains either a powdered or liquid drug 24. The syringe 10 typically contains diluent for reconstituting the vial contents 24.

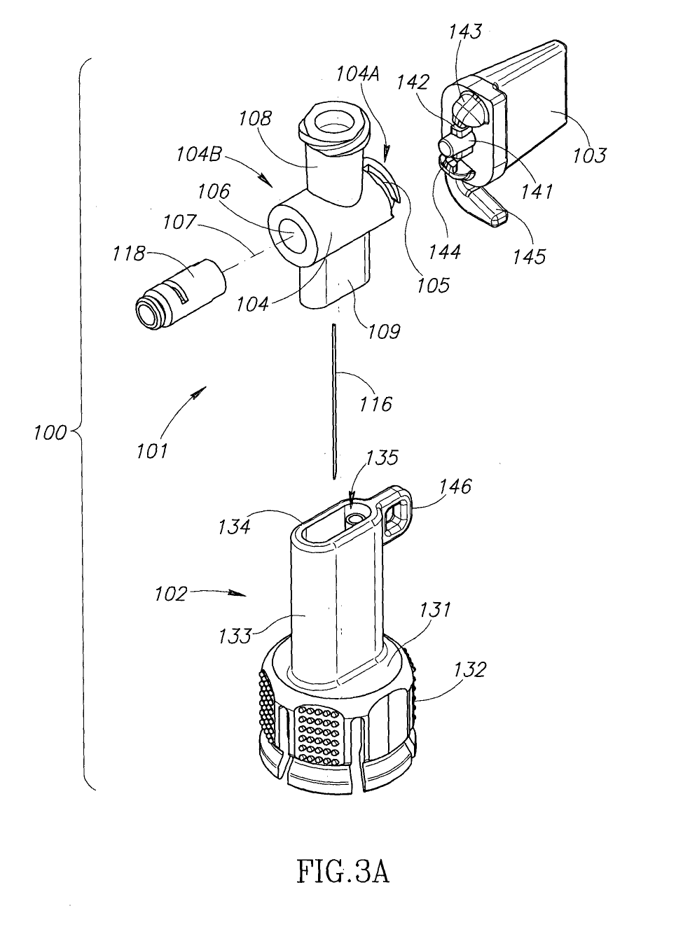

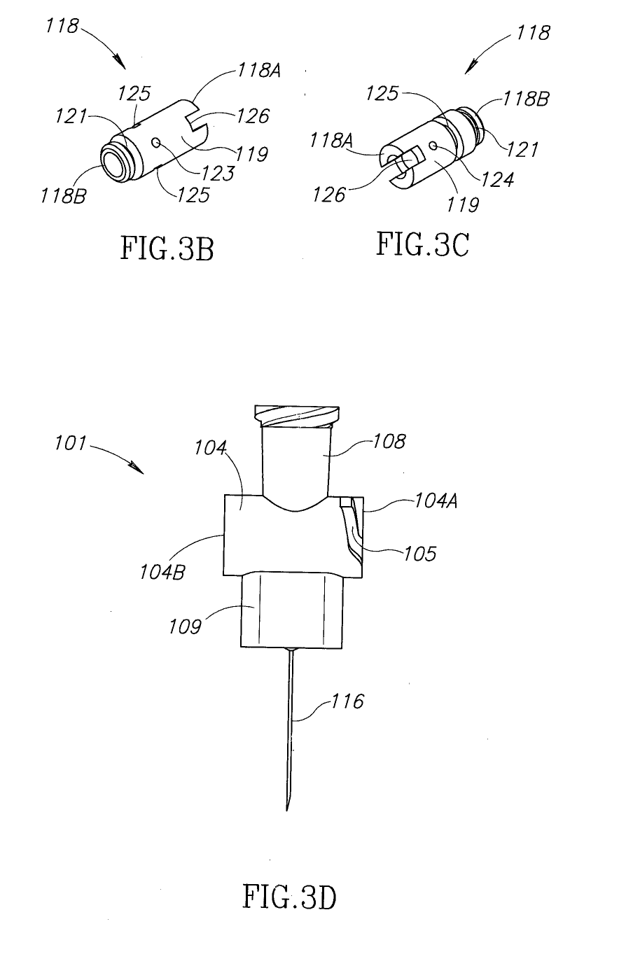

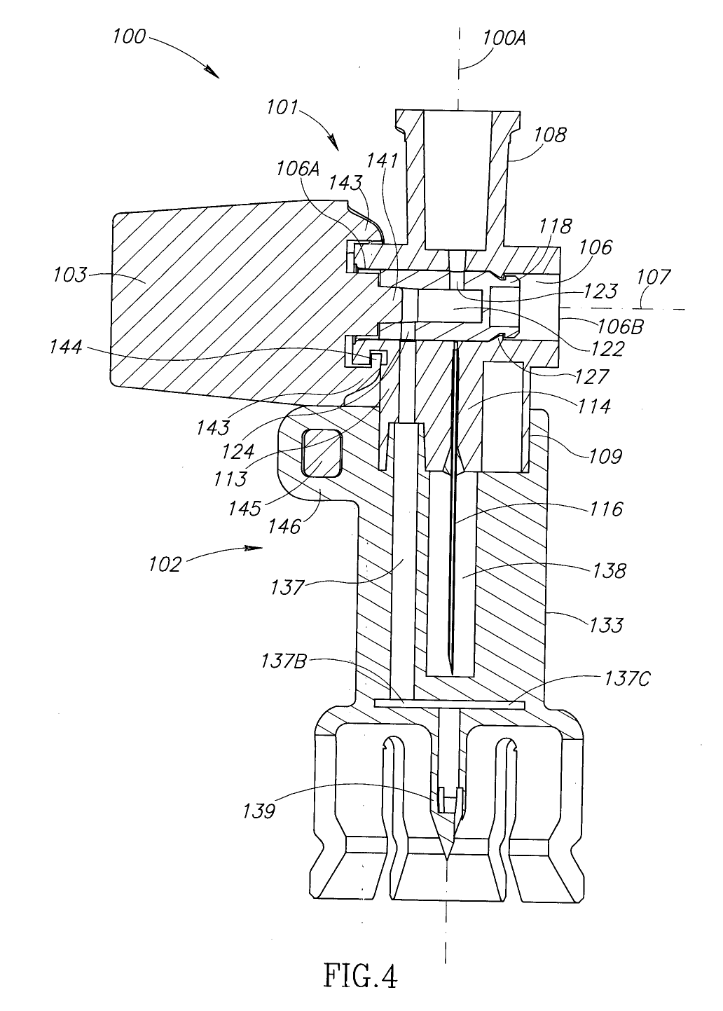

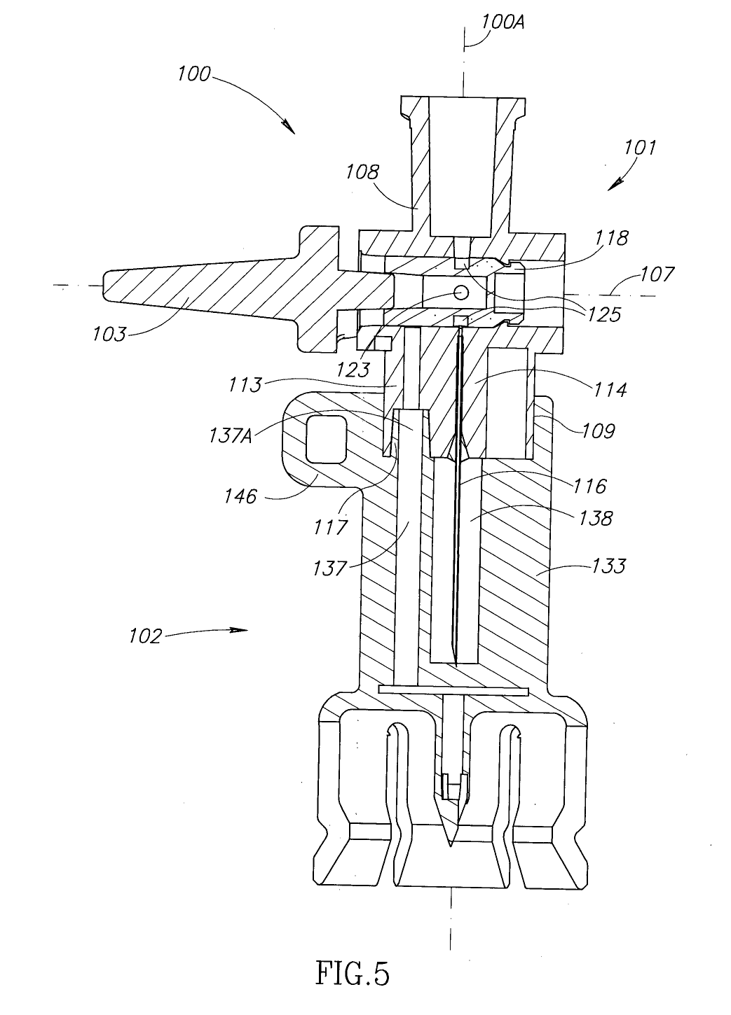

FIGS. 2 to 5 show the inline liquid drug medical device 100 has a longitudinal device axis 100A and includes a housing 101, a vial adapter 102 removably coupled on the housing 101, and a manually operated actuator 103 rotated from an initial liquid drug reconstitution position (see FIGS. 4A and 4B) to a subsequent liquid drug administration position (see FIGS. 5A and 5B) for operating the device 100. The housing 101 includes a generally cylindrical central body 104 transverse to the device axis 100A. The central body 104 has a proximal central body end 104A and a distal central body end 104B. The proximal central body end 104A is formed with pair of opposite screw threads 105 for screw thread mounting the actuator 103 on the housing 101. The central body 104 has a cylindrical throughgoing bore 106 with a bore axis 107 transverse to the device axis 100A. The bore 106 has a proximal bore end 106A and a distal bore end 106B corresponding with the proximal central body end 104A and the distal central body end 104B. The housing 101 includes a syringe port 108 constituting a first port in flow communication with the throughgoing bore 106 and a port manifold 109 on opposite sides of the central body 104 along the device axis 100A.

The syringe port 108 is in the form of a female Luer connector intended for receiving the syringe's male Luer lock connector 13 and is co-directional with the device axis 100A and co-axial therewith. The port manifold 109 has an elliptical cross-section as part of a keying arrangement for securing the vial adapter 102 relative to the housing 101. The port manifold 109 includes a second port 113 and a third port 114 both in flow communication with the throughgoing bore 106. The second port 113 and the third port 114 are co-directional with the device axis 100A and the third port 114 is co-axial therewith and therefore co-axial with the syringe port 108. The third port 114 is preferably fitted with a needle 116, a spray nozzle, and the like, for liquid drug administration purposes. The second port 113 is preferably recessed with respect to the third port 114 thereby forming a manifold cavity 117 as an additional feature of the keying arrangement.

The housing 101 includes a cylindrical flow control member 118 for rotary movement inside the throughgoing bore 106 about an axis of rotation co-axial with the bore axis 107 between an initial first flow control position for establishing flow communication between the syringe port 108 and the second port 113 for liquid drug reconstitution purposes and a subsequent second flow control position for establishing flow communication between the syringe port 108 and the third port 114 for liquid drug administration purposes. The flow control member 118 includes an open proximal end 118A, a sealed distal end 118B, and a peripheral cylindrical surface 119 formed with a circumferential recess 121 towards its sealed distal end 118B. The proximal end 118A is formed with an inner lumen 122 having a first aperture 123 and a second aperture 124 on opposite sides of the peripheral cylindrical surface 119. The second aperture 124 is displaced along the peripheral cylindrical surface 119 relative to the first aperture 113 such that the first aperture 123 is in flow communication with the syringe port 108 and the second aperture 124 is in flow communication with the second port 113 in the flow control member 118's first flow control position. The flow control member 118 is also formed with a semi-cylindrical conduit 125 formed on the peripheral cylindrical surface 119 opposite to the first aperture 123 such that the semi-cylindrical conduit 125 is in flow communication with the syringe port 108 and the third port 114 in the flow control member 118's second flow control position. The flow control member 118 is preferably designed such that it requires a quarter clockwise turn to urge it from its initial first flow control position to its second flow control position. The proximal end 118A is formed with a key slot 126 for engagement by the manually operated'actuator 103.

The throughgoing bore 106 has a uniform circular cross section therealong except for an area in relatively close proximity to its distal bore end 106B which is formed with an inward annular projection 127 for snap fit in the circumferential recess 121 for preventing possible sliding linear movement of the flow control member 118 inside the throughgoing bore 106. Alternatively, the circumferential recess 121 and matching projection 127 may be formed along a different section of the throughgoing bore 106, more proximal or distal from its distal end 106B.

The vial adapter 102 includes a skirt 129 with a top surface 131 and downward depending flex members 132 for snap fitting onto the vial 20. The vial adapter 102 includes an elongated upright stem 133 terminating in a stem tip 134 with a stem tip cavity 135 for fittedly coupling onto the port manifold 109. The stem tip cavity 135 is elliptically shaped, similarly to the cross-sectional shape of the port manifold 109 as part of the keying arrangement. The stem 133 includes a fluid conduit 137 with a proximal end 137A for sealing insertion in the manifold cavity 117 for sealed fluid communication with the second port 113 on coupling the vial adapter 102 on the housing 101. The stem 133 includes a bore 138 for receiving the needle 116 on coupling the vial adapter 102 on the housing 101. The fluid conduit 137, which is not co-axial with the device axis 100A, fluidly connects at a distal end 137B to a puncturing cannula 139 co-axial with the device axis 100A through a fluid interconnect conduit 137C. The puncturing cannula 139 serves to puncture the vial stopper 22 on its positive insertion into the vial adapter 102, and extends slightly therebeyond so that on inverting the vial 20 its nearly entire reconstituted liquid drug contents can be aspirated therefrom through the puncturing cannula 139.

The actuator 103 includes a proximal actuator end 103A facing towards the device axis 100A and a distal actuator end 103B facing away therefrom. The proximal actuator end 103A is formed with a cylindrical sealing projection 141 for sealing insertion into the proximal flow control member end 118A to seal the lumen 122. The proximal actuator end 103A is formed with a pair of keys 142 on opposing sides of the sealing projection 141 for engaging the key slot 126 such that rotation of the actuator 103 correspondingly rotates the flow control member 118.

The actuator 103 is removably mounted on the proximal central body end 104A by two actuator fasteners 143 on opposing sides of the sealing projection 141. The actuator fasteners 143 are formed with inwardly directed projections 144 facing towards the sealing projection 141 for engaging the screw threads 105. The actuator fasteners 143 engage the proximal central body end 104A in an initial liquid drug reconstitution position associated with the flow control member 118's first flow control position. The actuator fasteners 143 unscrew from the proximal central body end 104A on quarter clockwise rotation of the actuator 103 to its subsequent liquid drug administration position associated with the flow control member 118's second flow control position, thereby enabling removal of the actuator 103 in a direction along the bore axis 107.

The actuator 103 also includes a safety hook 145 for catching a safety catch 146 in the vial adapter 102 as part of a safety mechanism for preventing the vial adapter 102 being detached from the housing 101 while the actuator 103 is in the initial liquid drug reconstitution position. The safety hook 145 detaches from the safety catch 146 when the actuator 103 is rotated from its initial liquid drug reconstitution position to it subsequent liquid drug administration position, thereby enabling removal of the vial adapter 102 in a direction along the device axis 100A.

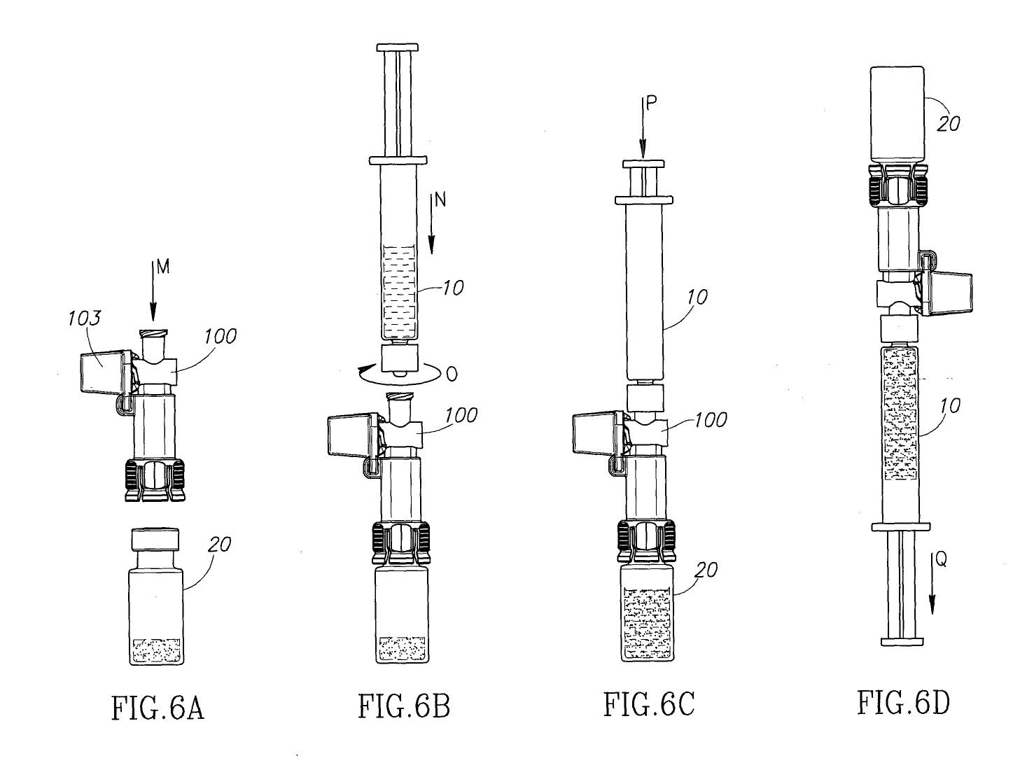

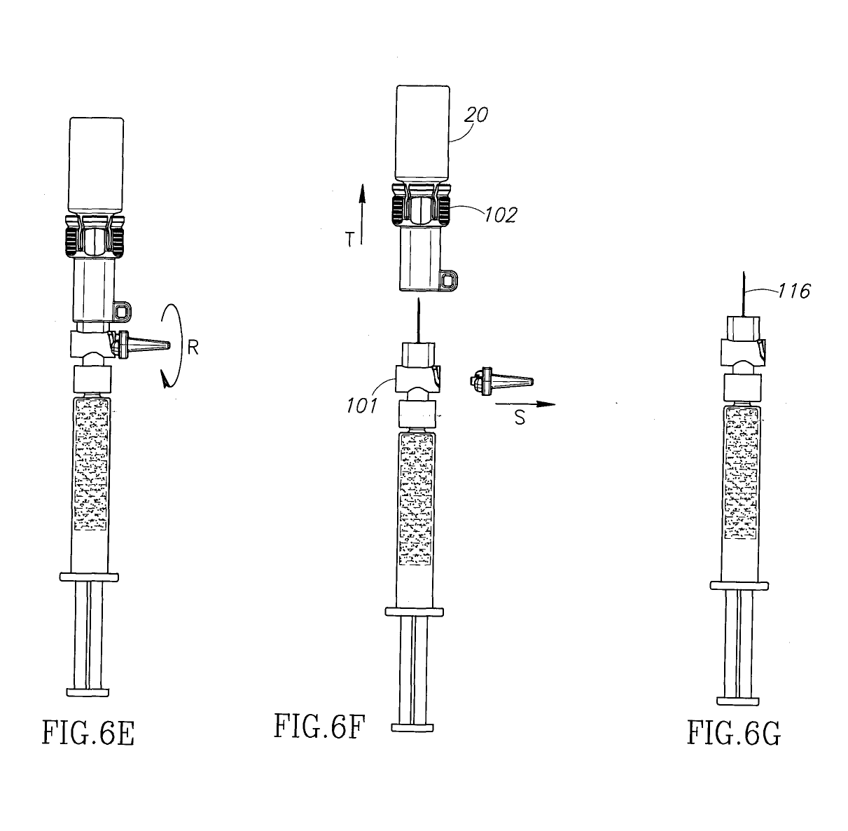

FIGS. 6A to 6G show the use of the inline liquid drug medical device 100 for liquid drug reconstitution and administration purposes as follows:

FIG. 6A shows the device 100 is in its initial first flow control position for liquid drug reconstitution and a user mounting the device 100 on a vial 20, as indicated by arrow M. The actuator 103 is in the initial liquid drug reconstitution position and the flow control member 118 is in its initial flow control position for liquid drug reconstitution purposes.

FIG. 6B shows the user approximating the syringe 10 towards the device 100, as indicated by arrow N, and screw threading the syringe 10 onto the device 100, as indicated by arrow O.

FIG. 6C shows the user injecting the syringe's contents into the vial 20, as indicated by arrow P. The user agitates the assemblage for reconstituting the liquid drug.

FIG. 6D shows the user inverting the assemblage and aspirating the reconstituted liquid drug contents into the syringe 10, as indicated by arrow Q. The actuator 103 is at all times in the initial liquid drug reconstitution position.

FIG. 6E shows the user rotating the actuator 103 to the subsequent liquid drug administration position for rotating the flow control member 118 to its subsequent second flow control position in which the syringe port 108 is in flow communication with the third port 114, as indicated by arrow R.

FIG. 6F shows the user removing the actuator 103, as indicated by arrow S, and pulling the vial adapter 102 with the spent vial 20 from the housing 101 for exposing the needle 116, as indicated by arrow T, thereby enabling administration of the liquid drug (see FIG. 6G).

While the invention has been described with respect to a limited number of embodiments, it will be appreciated that many variations, modifications, and other applications of the invention can be made within the scope of the appended claims. For example, the cross-sectional shape of the port manifold 109 and of the stem tip cavity 135 as part of the keying arrangement which may be a triangle, a rectangle, and the like, or combinations thereof, and which may include portions of the elliptical shape shown. Another example, can be that the flow control member 118 employs an external conduit for flow connection between the syringe port 108 and the second port 113 and an internal conduit for flow connection between the syringe port 108 and the third port 114. Still another example, the third port 114 may be recessed relative to the second port 113 so that the second port 113 protrudes into the fluid conduit member 137 and the bore 138 protrudes into a third port cavity.

1. An inline liquid drug medical device for use with a source of physiological solution and a medicinal vessel for reconstitution and administration of a liquid drug, the device having a longitudinal device axis, and comprising:
(a) a housing having a first port for fluid connection with the source of physiological solution, a second port for fluid connection with the medicinal vessel, a third port for liquid drug administration, said first port, said second port and said third port being co-directional with the device axis, and a bore with a bore axis transverse to the device axis and in flow communication with said first port, said second port and said third port;
(b) a flow control member rotatable in said bore about an axis of rotation co-axial with said bore axis from an initial first flow control position for establishing flow communication between said first port and said second port for liquid drug reconstitution purposes to a subsequent second flow control position for establishing flow communication between said first port and said third port for liquid drug administration purposes;
(c) a manually operated actuator having an initial liquid drug reconstitution position corresponding with said first flow control position and a subsequent liquid drug administration position corresponding to said second flow control position, said actuator rotating said flow control member from said first flow control position to said second flow control position on manually rotating said actuator from said initial liquid drug reconstitution position to said liquid drug administration position; and
(d) a vial adapter for snap fitting onto the medicinal vessel and including a fluid conduit member with a proximal end in flow communication with said second port and a distal end in flow communication with a puncturing cannula extending into the medicinal vessel on its attachment to said vial adapter, and said vial adapter being removably attached to said housing along a line of detachment co-directional with the device axis.

2. The device according to claim 1 wherein said manually operated actuator is removable from said housing on said manual rotation from said initial liquid drug reconstitution position to said subsequent liquid drug administration position.

3. The device according to claim 2 wherein said manually operated actuator is initially screw thread mounted on said housing in said initial liquid drug reconstitution position and simultaneously unscrews from said housing on said manual rotation from said initial liquid drug reconstitution position to said subsequent liquid drug administration position.

4. The device according to claim 1, wherein said manually operated actuator includes a safety hook for initial catching a safety catch integrally formed with said vial adapter for preventing said vial adapter being removed from said housing in said initial liquid drug reconstitution position whereupon rotation of said manually operated actuator to said subsequent liquid drug administration position removes said safety hook from said safety catch thereby enabling detachment of said vial adapter from said housing in said subsequent liquid drug administration position along a line of detachment co-directional with the device axis.
